(19) United States
(12) Patent Application Publication
(10) Pub. No.: US 2025/0009389 A1
(43) Pub. Date: Jan. 9, 2025

(54) TISSUE PENETRATING DEVICE

(71) Applicant: Yevgeny SHPAIKHLER, Har Gilo (IL)

(72) Inventor: Yevgeny SHPAIKHLER, Har Gilo (IL)

(21) Appl. No.: 18/712,032

(22) PCT Filed: Nov. 15, 2022
(86) PCT No.: PCT/IL2022/051219

§ 371 Date: May 21, 2024

(30) Foreign Application Priority Data
Nov. 25, 2021 (IL) 288443

(51) Int. Cl. A61B 17/34; A61M 27/00

(52) U.S. Cl. CPC A61B 17/3494; A61B 17/3415; A61M 27/00

(57) ABSTRACT

A handheld body penetration device for controllably penetrating through a body tissue into a body cavity, and for self arresting the penetration upon reaching the body cavity. The device comprises an elongated leading member having a leading tip for penetrating the body tissue; an advancement mechanism operatively connected to the leading member configured to induce axial advancement of the leading tip within the body tissue; and an arresting arrangement configured to prevent axial advancement upon the leading tip reaching the body cavity.

### TECHNOLOGICAL FIELD

The presently disclosed subject matter relates to a body penetration device, and more particularly to a body penetration device for controllably penetrating a body tissue.

### BACKGROUND

Placing a drain tube into a body cavity of a patient for either diagnostic or therapeutic purposes, by accessing said body cavity via the percutaneous route, is a fairly common medical-surgical procedure.

In such procedure, a hollow drain tube is inserted by the operator within the desired anatomical space, i.e. a body cavity, which is naturally present or pathologically formed, and filled with either gas liquid.

The insertion can be facilitated by wrapping the drain tube around a penetrating member configured to penetrate through a body tissue into the body cavity.

Advancing a penetrating member through a body tissue, into a body cavity carries definite risks for the patient, due to overpenetration, and also due to uncontrolled penetration through the body tissue, which can cause damage to the healthy surrounding tissues and organs.

When the operator is an untrained individual, and/or when the environment in which the procedure takes place is inconvenient for the operator, the risk is even greater.

There exists a need for a body penetration device which can be operated by an untrained person, in an inconvenient environment, for example by an untrained or minimally trained soldier, in the battlefield during a combat, while minimizing the risk of over-penetration and/or uncontrolled penetration.

### GENERAL DESCRIPTION

Generally, the presently disclosed subject matter is related to a body penetration device and a body penetration kit comprising said body penetration device along with other elements for enhancing usability thereof. The aspects detailed below are related to various manners of operations of the same, especially by an untrained person, in the field, and without a need for any specific mounting arrangement. For instance, the body penetration device described herein can be a hand-held device, i.e., may not require a mounting arrangement and can be held by the untrained person (user) and operated with ease.

The various aspects described herein below are to be understood as implementable in any combinations, i.e., any or all of the features of one aspect can be used with any one of the other aspects.

According to a first aspect of the presently disclosed subject matter there is provided a handheld body penetration device for controllably penetrating through a body tissue into a body cavity, and for self arresting said penetration upon reaching said body cavity, said device comprising: an elongated leading member having a proximal portion, a distal portion, and a longitudinal axis extending therebetween, said distal portion having a leading tip for penetrating said body tissue; an advancement mechanism operatively connected to said leading member, having an actuator configured to be actuated to induce axial advancement of the leading tip in an advancement direction along the longitudinal axis to a predetermined extent, within the body tissue; and an arresting arrangement configured to prevent said axial advancement upon the leading tip reaching the body cavity.

The handheld body penetration device can be used by any untrained user to penetrate through a human body in emergency situations. For instance, in a battlefield during a combat, where no trained professional treatment may be available. The medical procedure including drainage of fluids from a body cavity using a drainage tube (such as a cannula), can be performed using the body penetration device described herein by an untrained user (not trained for medical procedures) such as a simple soldier which have gone through minimal medical training, if any. Unlike the conventional body penetration devices, in which the user directly applies force to the penetrating element, the presently described body penetration device includes an advancement mechanism which translates the (uncontrolled) force applied on the advancement mechanism into a controlled advancement of the penetrating element (leading member/tip). The application of force directly on the penetrating element may lead to injury on the instances of accidental or uncontrolled application of force, and thus require a trained professional for the operation of the device. On the other hand, the body penetration device described herein has an actuator, on which the force is applied (manually or by a machine), and the actuator translates that force into advancement of the leading tip in a controlled manner, through a body tissue into the body cavity.

In order to translate the force into said advancement in a controlled manner, the advancement mechanism can be configured to advance the leading tip in discrete incremental steps. The actuator can be configured to be repeatedly actuated to advance the leading tip by a single increment with each actuation.

The actuator can be spaced apart from the leading member so as to avoid the force being applied directly to the leading member. In some examples, the actuator can be spaced apart from the longitudinal axis, such that the application of force may not be co-axial with the longitudinal axis. The actuator can be configured to displace along an actuator path upon said application of force, said actuator path being spaced from the longitudinal axis. In other examples, the actuator path can be coaxial with the longitudinal axis.

The advancement mechanism can comprise a handle to be held by a hand of the user, having a proximal handle portion being most proximal to the leading member, an opposite distal handle portion, and a handle axis extending therebetween, said handle axis being angled with respect to the longitudinal axis. The actuator can comprise a lever trigger configured to be operated by the hand of the user while holding the handle. The handle and the actuator so configured facilitates an easy handheld usage of the device without the need of a mounting arrangement. The handle axis being angled with respect to the longitudinal axis contributes to the application of force being spaced apart from the longitudinal axis. The actuator can be configured to be actuated by a force applied by the hand of the user, and said advancement mechanism can be configured to translate said force to said axial advancement of said leading tip.

The body penetration device can comprise a stationary member configured to remain stationary with respect to the body, and a movable member including at least the leading member being movable with respect to the stationary member, during said penetration of the leading tip thereinto. The stationary member can comprise a leaning surface configured to lean on the body during penetration of the leading tip, through which said leading member extends, at least during operation of the device. The leaning surface leaning on the body ensures a better stability to the device during use thereof. In some examples, the leading member may extend through the leaning surface, during penetration, and advance thereacross, along with the drain tube.

In addition to the controlled advancement of the leading tip into the body, the body penetration device described herein provides an additional layer of safety to the operation of the device, in that, the device is configured for self-arresting the advancement as soon as the leading tip reaches the body cavity. Thus, the device is configured to prevent over-penetration of the leading tip inside the human body. To this effect, the arresting arrangement can be switchable between a release state in which the arresting member allows the operation of the advancement mechanism and an arresting state in which the arresting arrangement arrests the operation of the advancement mechanism. The arresting arrangement can be configured to remain at its release state as long as the leading tip is urged against a direction of its advancement by an opposing force greater than a predetermined threshold, and switch into its arresting state in response to that opposing force falling below the predetermined threshold. The opposing force is applied by the body and/or the body tissue on the leading tip resisting the advancement thereof. The opposing force is greater than the predetermined threshold until the leading tip advanced against the body tissue, and falls below the predetermined threshold as soon as the leading tip reaches the body cavity, thereby indicating that the leading tip has reached the body cavity. The arresting arrangement automatically and instantly switches to the arresting state thereof in response to such indication.

The arresting arrangement can comprise an arresting member configured to, in the arresting state of the arresting arrangement, inter-lockingly connect the movable member with the stationary member and, in the release state of the arresting arrangement, disconnect the movable member from the stationary member.

The arresting arrangement can comprises a biasing member operatively connected to the arresting member and having a biasing force constantly biasing the leading tip in the direction of the advancement of the leading tip along the longitudinal axis against said opposing force, said biasing member being switchable between a first state corresponding to the release state of the arresting arrangement, in which the opposing force is greater than the biasing force, and a second state corresponding to the arresting state of the arresting arrangement, in which the opposing force is lesser than the biasing force. The biasing member can be connected to said leading member such that said opposing force which the leading tip encounters is transmitted through the leading member to the biasing member to cause the biasing member to switch between first and second states thereof. The biasing force of the biasing member can be adjustable by manipulating the biasing member.

Once the leading tip has reached the body cavity, the leading member can be retracted using a resetting mechanism configured to facilitate retraction of the leading member from the body.

The body penetration device can comprise a drain tube mounting member surrounding said leading member having an external surface configured to be wrapped with a drainage tube, and configured to be advanced together with the leading tip thereof into the body. Such structure allows simultaneously insertion of the tube with the leading tip into the body.

The external surface can be configured to facilitate deployment of said drainage tube, once the tip has reached the cavity, between said body cavity and an exterior of the body for withdrawing fluids from the body cavity. For instance, an end of the drainage tube coincides with the leading tip and reaches the body cavity therealong. When the leading member is retracted, the minimal friction between the drainage tube and the drain tube mounting member causes the drainage tube not to be retracted and be deployed with the end of the drainage tube in the body cavity and the other end being external to the body. The drainage tube can then be used for draining fluids from the body cavity.

In some examples, during said switching of the biasing member said leading member moves with respect to said drain tube mounting member between a rear position corresponding to the first state of the biasing member, and a front position corresponding to the second state of the biasing member. In other examples, the leading member can move between the rear position and the front position with respect to an element of the advancement mechanism that is operatively connected to the leading member.

According to a second aspect of the presently disclosed subject matter there is provided a body penetration kit comprising: the handheld body penetration device according to the first aspect; and a drainage tube configured to be deployed between a body cavity and an exterior of the body for withdrawing fluids therefrom. The drainage tube can be wrapped upon an external surface of the leading member. In other examples, when the device comprises the drain tube mounting member, the drainage tube can be wrapped upon an external surface thereof. The kit can further comprise a sterile bag accommodating the handheld body penetration device and the drainage tube in a sterile manner. The kit can be a single use kit and the sterile bag can be a disposable bag.

### EMBODIMENTS

A more specific description is provided in the Detailed Description whilst the following are non-limiting examples of different embodiments of the presently disclosed subject matter. It should be appreciated that Embodiments 1 to 19, correspond to the first aspect of the presently disclosed subject matter; and Embodiments 20 to 22, correspond to the second aspect of the presently disclosed subject matter;
1. A handheld body penetration device for controllably penetrating through a body tissue into a body cavity, and for self arresting said penetration upon reaching said body cavity, said device comprising:
a. an elongated leading member having a proximal portion, a distal portion, and a longitudinal axis extending therebetween, said distal portion having a leading tip for penetrating said body tissue;
b. an advancement mechanism operatively connected to said leading member, having an actuator configured to be actuated to induce axial advancement of the leading tip in an advancement direction along the longitudinal axis to a predetermined extent, within the body tissue; and
c. an arresting arrangement configured to prevent said axial advancement upon the leading tip reaching the body cavity.
2. The handheld body penetration device according to Embodiment 1, wherein the advancement mechanism is configured to advance the leading tip in discrete incremental steps.
3. The handheld body penetration device according to Embodiment 2, wherein the actuator is configured to be repeatedly actuated to advance the leading tip by a single increment with each actuation.
4. The handheld body penetration device according to any one of the preceding embodiments, wherein the actuator is spaced apart from the leading member.
5. The handheld body penetration device according to any one of the preceding embodiments, wherein the actuator is spaced apart from the longitudinal axis.
6. The handheld body penetration device according to any one of the preceding embodiments, wherein the actuator is configured to displace along an actuator path upon said application of force, said actuator path being spaced from the longitudinal axis.
7. The handheld body penetration device according to any one of the preceding embodiments, wherein the advancement mechanism comprises a handle to be held by a hand of the user, having a proximal handle portion being most proximal to the leading member, an opposite distal handle portion, and a handle axis extending therebetween, said handle axis being angled with respect to the longitudinal axis.
8. The handheld body penetration device according to Embodiment 7, the actuator comprises a lever trigger configured to be operated by the hand of the user while holding the handle.
9. The handheld body penetration device according to any one of the preceding embodiments, wherein the actuator is configured to be actuated by a force applied by the hand of the user, and said advancement mechanism is configured to translate said force to said axial advancement of said leading tip.
10. The handheld body penetration device according to any one of the preceding embodiments, wherein the device comprises a stationary member configured to remain stationary with respect to the body, and a movable member including at least the leading member being movable with respect to the stationary member, during said penetration of the leading tip thereinto.
11. The handheld body penetration device according to Embodiment 10, wherein the stationary member comprises a leaning surface configured to lean on the body during penetration of the leading tip, through which said leading member extends, at least during operation of the device.
12. The handheld body penetration device according to any one of the preceding embodiments, wherein the arresting arrangement is switchable between a release state in which the arresting member allows the operation of the advancement mechanism and an arresting state in which the arresting arrangement arrests the operation of the advancement mechanism.
13. The handheld body penetration device according to Embodiment 10, wherein the arresting arrangement is configured to remain at its release state as long as the leading tip is urged against a direction of its advancement by an opposing force greater than a predetermined threshold, and switch into its arresting state in response to that opposing force falling below the predetermined threshold.
14. The handheld body penetration device according to Embodiment 12 or 13 when dependent on Embodiment 10, wherein the arresting arrangement comprises an arresting member configured to, in the arresting state of the arresting arrangement, inter-lockingly connect the movable member with the stationary member and, in the release state of the arresting arrangement, disconnect the movable member from the stationary member.
15. The handheld body penetration device according to Embodiment 14, when dependent on Embodiment 13, wherein the arresting arrangement comprises a biasing member operatively connected to the arresting member and having a biasing force constantly biasing the leading tip in the direction of the advancement of the leading tip along the longitudinal axis against said opposing force, said biasing member being switchable between a first state corresponding to the release state of the arresting arrangement, in which the opposing force is greater than the biasing force, and a second state corresponding to the arresting state of the arresting arrangement, in which the opposing force is lesser than the biasing force.
16. The handheld body penetration device according to Embodiment 15, wherein said biasing member is connected to said leading member such that said opposing force which the leading tip encounters is transmitted through the leading member to the biasing member to cause the biasing member to switch between first and second states thereof.
17. The handheld body penetration device according to any of the preceding embodiments, further comprising a drain tube mounting member surrounding said leading member having an external surface configured to be wrapped with a drainage tube.
18. The handheld body penetration device according to Embodiment 18, wherein said external surface is configured to facilitate deployment of said drainage tube between said body cavity and an exterior of the body for withdrawing fluids from the body cavity.
19. The handheld body penetration device according to Embodiment 17 or 18, when depending on Embodiment 16, wherein during said switching of the biasing member said leading member moves with respect to said drain tube mounting member between a rear position corresponding to the first state of the biasing member, and a front position corresponding to the second state of the biasing member.
20. A body penetration kit comprising:
a. the handheld body penetration device according to any one of the preceding embodiments; and
b. a drainage tube configured to be deployed between a body cavity and an exterior of the body for withdrawing fluids therefrom.
21. The kit according to Embodiment 20, wherein the drainage tube is wrapped upon an external surface of the leading member.
22. The kit according to Embodiment 20 or 21, further comprising a sterile bag accommodating the handheld body penetration device and the drainage tube in a sterile manner.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A illustrates a side perspective view of a body penetration device, according to an example of the presently disclosed subject matter, in its non-advanced and released state;

FIG. 1B illustrates a side view of the body penetration device of FIG. 1A in the same state as illustrated in FIG. 1A;

FIG. 1C illustrates a cross-section taken along line A-A in FIG. 1B;

FIG. 1D illustrates a side view of the body penetration device of FIG. 1A in its advanced and released state;

FIG. 1E illustrates a cross-section taken along line B-B in FIG. 1D;

FIG. 1F illustrates the same view as illustrated in FIG. 1E, with the body penetration device in its advanced and arrested state; and

FIG. 2 illustrates a body penetration kit comprising the body penetration device of FIG. 1A.

### DETAILED DESCRIPTION OF EMBODIMENTS

The below detailed description relates to a handheld body penetration device and a kit comprising the same along with other elements to enhance usability thereof. The below described body penetration device can be used without the need for a trained medical practitioner or a professional, and even without any specific mounting arrangement. The body penetration device can be held by a user in a single hand and can be advanced in a controlled manner into a human body. The device has a leading tip that penetrates through body tissue to reach a body cavity which is to be accessed for a treatment. The treatment can be drainage of fluids from the body cavity. The device has an advancement mechanism that upon actuation by the user causes the leading tip to advance into the human body when the device is in its operational position. One of the major advantages of the body penetration device described herein is that the device can be held in the hands of a user and can be operated even by an untrained user, while minimizing the risk of hurting the patient on which the device is being used. For instance, the advancement mechanism advances the leading tip in a controlled manner such that the penetration is smooth and consistent even if the user accidently and/or due to lack of experience applies an uncontrolled or undue amount of force. Further, the device is capable of automatic self-arresting upon the leading tip reaching the body cavity thereby avoiding over-penetration. Upon the self-arrest, the leading tip cannot be penetrated further irrespective of the force applied on the device.

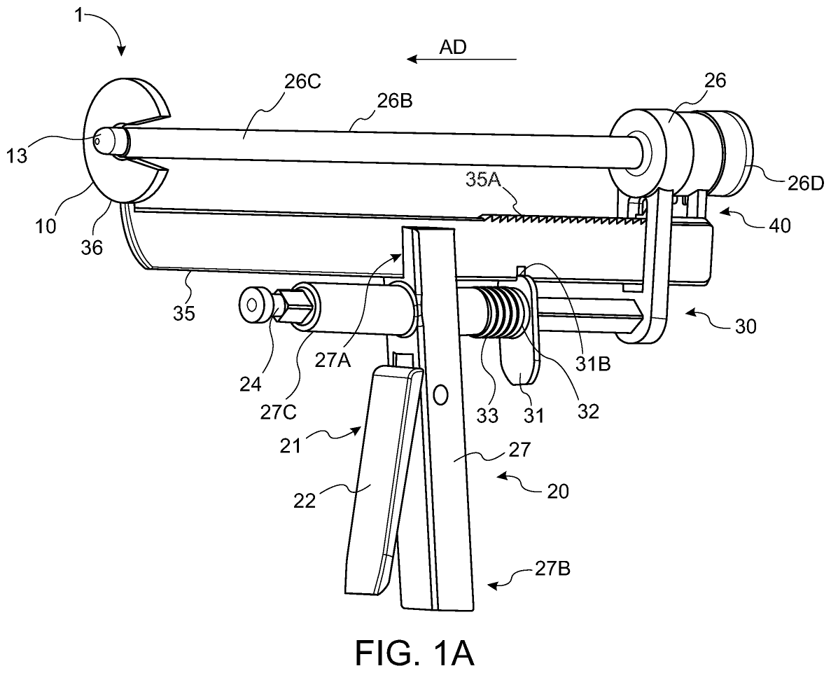

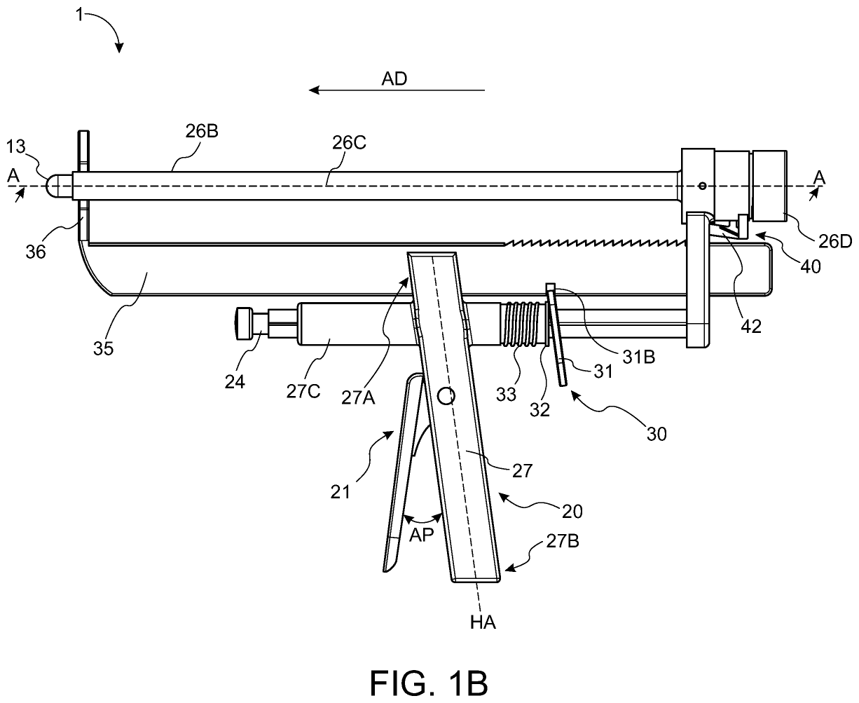

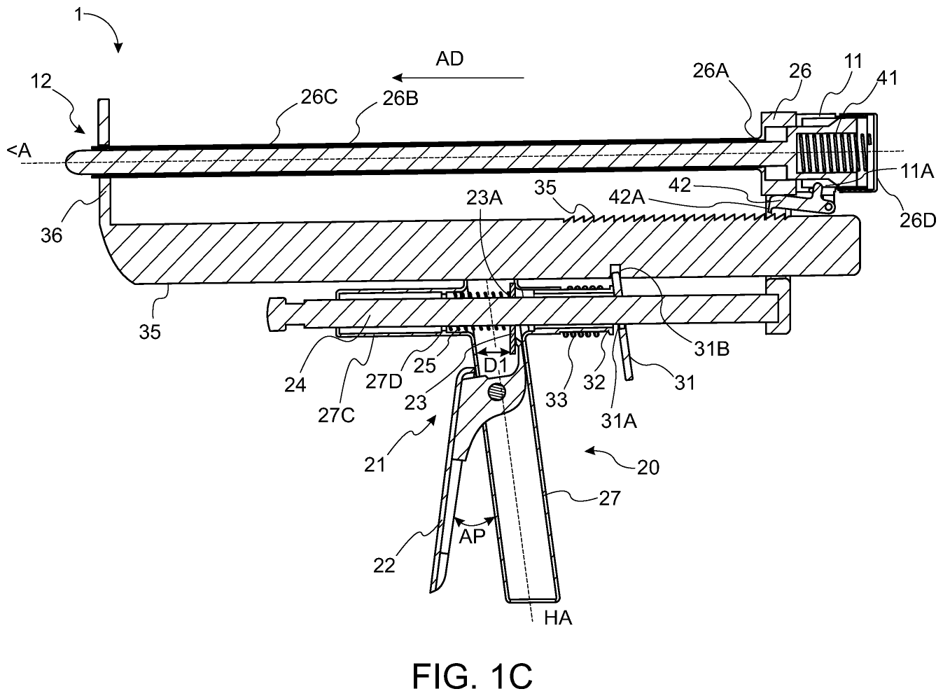

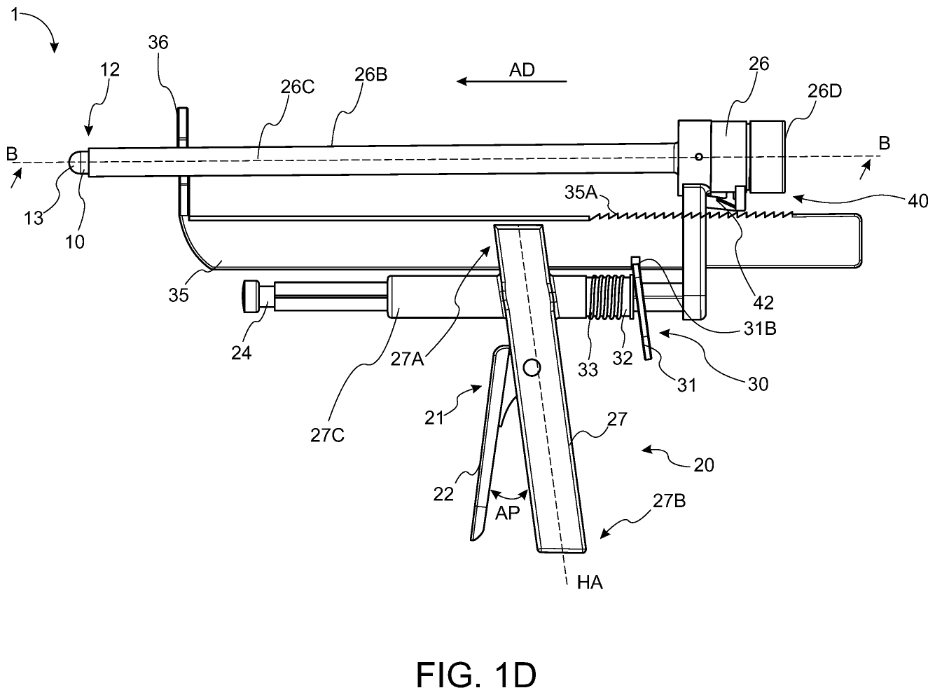

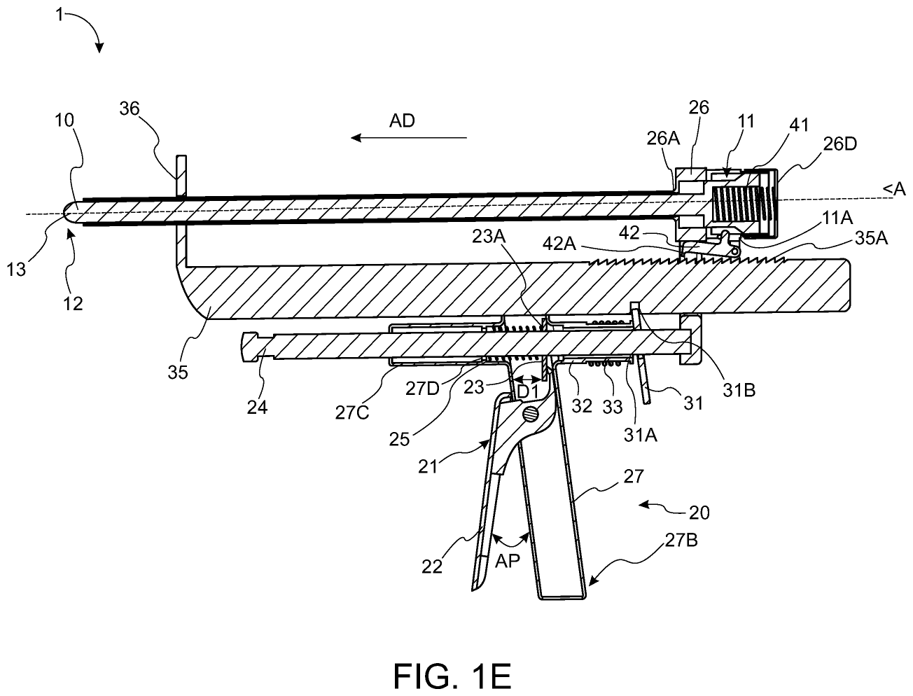

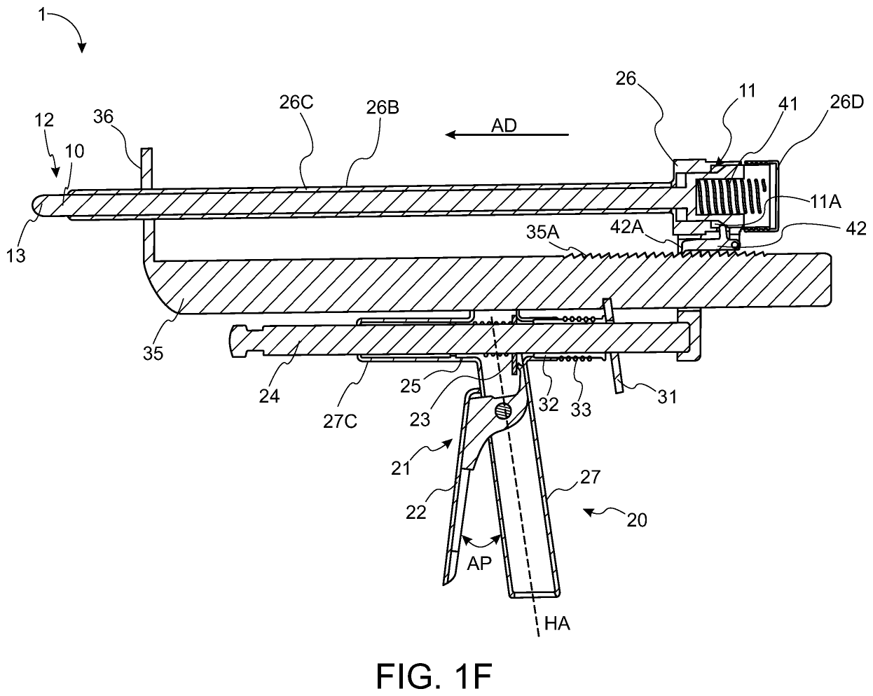

Attention is now directed to FIGS. 1A to 1F of the drawings illustrating a body penetration device 1 having an elongated leading member 10 extending along a longitudinal axis LA, an advancement mechanism 20, and an arresting arrangement 40. The leading member 10 has a proximal portion 11 and a distal portion 12 including a leading tip 13 to be positioned towards a body (not shown) during operation of the device 1. The advancement mechanism 20 comprises an actuator 21 operatively connected to the leading member 10 such that when the actuator is actuated, it induces an axial advancement of the leading tip 13 into the body in an advancement direction AD to a predetermined extent (explained in detail later herein below). The arresting arrangement 40 is configured to mechanically sense that the leading tip 13 has reached a body cavity, and arrests the operation of the advancement mechanism 20 to prevent the axial advancement of the leading tip 13 in the advancement direction AD.

As can be best seen in FIG. 1C, the actuator 21 includes a lever trigger 22 to be actuated by an application of force, which in the illustrated example can be applied by a hand of the user holding the device 1. In some examples, the force can be applied by a machine such as a robot, motor, etc. In the illustrated example, the actuator 21 follows an actuator path AP, which is distant from the longitudinal axis LA. In other words, the actuator 21 is spaced apart from the longitudinal axis LA such that the application of force for advancing the leading tip is not coaxial with the leading member. In some examples, the actuator can be positioned such that the application of force for advancing the leading tip can be coaxial with the leading member and the longitudinal axis.

The advancement mechanism 20 further includes an advancing plate 23 operatively coupled to the actuator 21 and an intermediate rod 24 operatively coupled to the advancing plate 23. The intermediate rod 24 is configured to be advanced by the advancing plate 23 upon action of the actuator 21 on the advancing plate 23, yet once force is removed from the actuator 21, the advancing plate 23 can slidingly biased into a rear position by an advancing spring 25, to be ready for another advancement of the intermediate rod 24. The actuator 21, when actuated, displaces the advancing plate 23 into a front position against the bias of the advancing spring 25, which biases the advancing plate 23 back to its rear position when the force applied on the actuator is removed. The advancement mechanism 20 further includes a holder 26 connected to the intermediate rod 24 and configured to firmly hold the leading member 10 in a rear position (seen in FIGS. 1A to 1E) and a front position (seen in FIG. 1F) explained in detail later herein below. In some examples, the intermediate rod 24 and the holder 26 can be manufactured as a single unit. The advancing plate 23, the intermediate rod 24, and the holder 26 together operatively connecting the actuator 21 to the leading member 10 such that actuation of the actuator 21 induces the advancement of the leading member 10, and hence the leading tip 13, in the advancement direction AD along the longitudinal axis LA by a predetermined extent D1.

The advancing plate 23 comprises a through-hole 23A and the intermediate rod 24 passes therethrough. The dimensions and shape of the through-hole 23A and that of the intermediate rod 24 corresponding thereto are such that when the advancing plate 23 is perpendicular to the intermediate rod 24, the through-hole 23A aligns with the intermediate rod 24 so as to allow the intermediate rod 24 and the advancing plate 23 to freely displace with respect to each other. When the advancing plate 23 is inclined with respect to the intermediate rod 24, the through-hole 23A becomes fixated with the intermediate rod 24 such that displacement of the advancing plate 23 causes the displacement of intermediate rod 24. The advancement mechanism 20 has a handle 27 having a proximal handle portion 27A positioned proximal to the leading member 10, an opposite handle distal portion 27B, and a handle axis HA angled with respect to the longitudinal axis LA. A user can hold the device 1 by handle 27 and can actuate the actuator 21 using one or more fingers of the same hand holding the device 1, or the different hand. The proximal handle portion 27A has a hollow elongate member 27C extending in a direction along the longitudinal axis LA. The intermediate rod 24 is positioned inside and configured to be displaced within the hollow elongate member 27C. The intermediate rod 24 has the advancing spring 25 wrapped therearound. The advancing spring 25 is biased between the advancing plate 23 and a stopper 27D mounted inside the hollow elongate member 27C.

The advancing plate 23 is normally at its rear position within the hollow elongate member 27C, and perpendicular to the intermediate rod 24. When the actuator 21 is actuated by application of force on the lever trigger 22, the actuator 21 pushes the advancing plate 23 at one of its ends causing the advancing plate 23 to become inclined with respect to the intermediate rod 24. Then, a further push of the actuator 21 on the advancing plate 23 causes the advancing plate 23 to displace into its front position, which displaces therealong the intermediate rod 24. The intermediate rod 24 being operatively connected to the leading member 10 via the holder 26 causes the leading member 10 and the leading tip 13 to advance in the advancement direction AD by a predetermined extent defined by the distance D1 between the rear and the front position of the advancing plate 23.

When the force is removed from the actuator 21, the advancing spring 25 causes the advancing plate 23 to be perpendicular to the intermediate rod 24, and then to slidingly displace into its rear position while not displacing the intermediate rod 24 backwards. Thus, the leading member 10 and the leading tip 13 is advanced by the predetermined extent D1 by a single action of force on the actuator 21. With every such action of force, the leading member 10 and the leading tip 13 is advanced by the predetermined extent D1. Thus, the leading tip 13 is penetrated into the human body in discrete incremental steps by repetitive actions on the actuator 23. The actuator 21 translates each action of force thereupon into the predetermined extent D1 of the advancement of the leading tip 13, i.e., in a controlled manner, irrespective of the amount of force applied in each such action.

In some examples, the advancing mechanism can be configured to advance the leading tip in a continuous advancement but in a controlled manner, for example, at a controlled speed irrespective of the amount of force applied at the actuator.

The body penetration device 1 further comprises a resetting mechanism 30 including a resetting plate 31 having a through-hole 31A. The resetting plate 31 is pivotable about a pivot point 31B, which in the illustrated example is at a stationary member (explained in detail later herein below). In some examples, the pivot point can be associated with the handle 27. The intermediate rod 24 passes through the through-hole 31A, and the dimensions and shape of the through-hole 31A and that of the intermediate rod 24 corresponding thereto are such that when the advancing plate 31 is perpendicular to the intermediate rod 24, the through-hole 31A aligns with the intermediate rod 24 so as to allow the intermediate rod 24 and the advancing plate 31 to freely displace with respect to each other. When the advancing plate 31 is inclined with respect to the intermediate rod 24, the through-hole 31A becomes fixated with the intermediate rod 24 such that displacement of the intermediate rod 24 is prevented through the through-hole 31A. The resetting mechanism 30 further has a resetting member 32 surrounding the intermediate rod 24, and positioned partially within the hollow elongate member 27C. A resetting spring 33 (also constituting a part of the resetting mechanism 30) surrounds the resetting member 32, and is biased between the resetting plate 31 and the hollow elongate member 27C. The resetting spring 33 normally biases the resetting plate 31 so as to be inclined with respect to the intermediate rod 24. During the advancement of the leading member 10, when the intermediate rod 24 is displaced by the advancing plate 23, the fixation between the resetting plate 31 and the intermediate rod 24 (due to the resetting plate 31 being inclined with respect to the intermediate rod 24) causes the resetting plate 31 to pivot about the pivot point 31B against the resetting spring 33 and become perpendicular to the intermediate rod 24. Thus, at this state, the intermediate rod 24 can displace through the through-hole 31A. When the intermediate rod 24 has been displaced by the predetermined extent and that displacement is complete, the biasing force of the resetting spring 33 causes the resetting plate 31 to be inclined with respect to the intermediate rod 24. Thus, the resetting plate 31 prevents the backward displacement of the intermediate rod 24. When the leading tip 13 needs to be retracted from the human body, i.e., the body penetration device needs to be reset, the resetting plate can be pushed manually (or by a machine) to pivot about the pivot point 31B and to become perpendicular to the intermediate rod 24, and then the intermediate rod 24, and the leading tip 13, can be retracted backwards along the same path through which it was advanced by the advancement mechanism 20.

Although the advancement mechanism 20 and the resetting mechanism 30 have been described above with the specific structure, it is to be understood herein that the advancement mechanism and/or the resetting mechanism can be achieved by a different, more or less complex, structure to achieve the same purpose of the controlled advancement and resetting of the leading tip.

The body penetration device 1 further comprises a stationary member 35 having a leaning member 36 through which the leading member 10 passes. The leaning member 36, best seen in FIG. 1A, is configured to lean against the human body when the device 1 is in operation. The leaning member 35 contributes to keeping the whole device I stable during operation. The leading member 10 passes through the leaning member 36 and moves with respect thereto, thereby constituting a moveable member of the body penetration device 1. In the illustrated example, the handle 27 is connected to the stationary member 35.

The holder 26 holds therewithin the proximal portion 11 of the leading member 10 such that the leading member 10 extends from a holder opening 26A and is displaceable between a rear position (as seen in FIG. 1F) and a front position (as seen in FIGS. 1A to 1E) with respect to the holder 26. The leading member 10 is surrounded by a drain tube mounting member 26B, which in the illustrated example constitutes a part of the holder 26. In other examples, the drain tube mounting member can be a separate element. The drain tube mounting member 26B has an external surface 26C configured to be wrapped with a drainage tube (not shown). The leading member 10 when advanced into the body tissue takes therealong the drain tube mounting member 26B along with the drainage tube, and when reaches the body cavity, positions a tip of the drainage tube within the body cavity. When the leading member 10 is retracted, the external surface 26C of the drain tube mounting member 26B slides with respect to the drainage tube thereby leaving the tip of the drainage tip within the body cavity and the device 1 can be removed. Thus, the drainage tube is deployed for drainage of fluids from the body cavity.

The arresting arrangement 40 comprises a biasing member 41 connected to the proximal portion 11 of the leading member 10 and constantly biasing the leading member 10, and thus the leading tip 13, towards the advancement direction AD. The biasing member 41 is positioned within the holder 26 and is biased between the proximal portion 11 of the leading member 10 and a holder lid 26D. The proximal portion 11, and the leading member 10, is displaceable between a front position and a rear position with respect to the holder 26 and in the illustrated example, with respect to the drain tube mounting member 26B. For instance, when the leading tip 13 is held against the body (at the beginning of the operation of the device 1) and/or the body tissue (during the operation of the device 1), the leading tip 13 experiences an opposing force (opposing the advancement of the leading tip 13) in a direction opposite to the advancing direction AD. Until the opposing force is greater than the biasing force of the biasing member 41, the opposing force maintains the biasing member 41 in its first state and the leading member 10 in its rear position with respect to the holder 26. When the device is positioned away from the body to be penetrated, the biasing force of the biasing member 41 pushes the leading member 10 into its front position. When the leading tip 13 is positioned against the body and/or the body tissue, namely, being pressed thereagainst by the hand of the user, the biasing member 41 retracts thereby bringing the leading member 10 into its rear position. As soon as the leading tip 13, after having penetrated through the body tissue, reaches the body cavity, the opposing force instantly falls. The biasing force applied by the biasing member 41 is adjusted to be greater than the opposing force applied within the cavity, such that upon reaching the body cavity, the biasing member 41 instantly displaces into its second state, thereby displacing the leading member 10 into its front position with respect to the holder 26, i.e., indicating/sensing that the leading tip 13 has reached the body cavity. The holder lid 26D is mounted to the holder 26 via threading arrangement, and the extent of threading is controllable to control the position of the holder lid 26D with respect to the holder 26, thereby controlling the biasing force of the biasing member 41. In other examples, the biasing force of the biasing member can be adjustable by other means to serve the same purpose of having adjustable biasing force or the predetermined threshold for the arresting arrangement to operate.

At this position of the leading tip 13 within the body cavity, it is preferable to prevent further advancement of the leading tip 13 into the body. The arresting arrangement 40 further comprises an arresting member 42 connected to the holder 26. The holder 26 is configured to advance along with the leading member 10 by the operation of the advancement mechanism 20, as described above. Thus, the holder 26, along with the leading member 10, constitutes the movable members of the body penetration device 1. The arresting member 42 being connected to the holder 26 also advances therealong by the operation of the advancement mechanism 20. The arresting member 42 is positioned, and displaces along the advancement direction, in proximity to the stationary member 35. The arresting member 42 is disconnected from the stationary member 35 and is retained in such position until the leading member is in its rear position. The proximal portion 11 of the leading member 10 comprises a manipulating member 11A configured to manipulate the arresting member 42 when the leading member 10 displaces into its front position with respect to the holder 26. This manipulation causes the arresting member to interlockingly connect to the stationary member 35 with the movable member, as can be seen in FIG. 1F. This interlocking arrests the movable member (the arresting member 42, the holder 26, the leading member 10, and the intermediate rod 24) with the stationary member 35, thereby preventing the advancement of the leading member 10. In other words, such interlocking arrests the operation of the advancement mechanism, in that, the advancement mechanism is inoperable, at this state of the arresting member 42, to advance the leading member 10.

In the illustrated example, the stationary member 35 comprises a toothed surface 35A and the arresting member 42 has a sharp end 42A, and the interlocking connection of the arresting member 42 with the stationary member 35 includes interlocking of the sharp end 42A with the toothed surface 35A, as can be best seen in FIG. 1F. In the illustrated example, the teeth 35A are inclined in a direction opposite the advancement direction AD, in such a way that when the arresting member 42 is interlocked therewith, the displacement of the arresting member 42 towards the advancement direction is arrested while the displacement of the arresting member 42 in the opposite direction is allowed. Thus, once the advancement of the leading member 10 is arrested, upon the leading tip 13 reaching the body cavity, the drain tube can be deployed thereat, and the device can be removed, optionally, the leading member can be retracted using the resetting mechanism.

It should be appreciated that the size of each tooth, and the distance between two adjacent apexes thereof, is smaller than the distance D1 in which the leading tip 13 advances in every increment. Such arrangement allows arresting even in a middle of an actuation action performed by a user, i.e., when the trigger is pressed to half-way.

In other examples, the teeth can have different shapes and structures to achieve the same purpose of arresting the operation of the advancement mechanism.

In operation of the body penetration device 1, the user holds the device 1 from handle 27 and leans the leaning surface 36 against the body to be penetrated. The body can have an incision already made at the location of the penetration, and thus the leading tip 13 needs not be sharp, although it can be tapered in the advancement direction. Also, as the device I does not operate on any principle requiring flow of blood into the device 1, thus the leading member 10 can be solid, thereby providing strength to the leading member 10, and effective transmission of the opposing force from the leading tip 13 to the biasing member 41. The leaning of the leaning surface 36 against the body causes the leading tip to be pressed against the body and thereby experience the opposing force, which when being greater than the biasing force of the biasing member 41 pushes the leading member into its rear position, thereby displacing the arresting arrangement 40 into its release state, at which the advancement mechanism 20 can be operated to advance the leading tip 13 into the body. Then, the advancement mechanism 20 is operated to advance the leading tip 13 into the body through the body tissue in a controlled manner, as detailed above. Until the leading tip 13 is positioned against the body or the body tissue, the opposing force remains smaller than the biasing force of the biasing member 41, the leading member 10 is at its front position, and the arresting member is in its arresting state. Upon the leading tip 13 being pressed against the body to induce an opposing force which is greater than the biasing force of the biasing member 41, the leading member 10 is pushed into its rear position and the arresting arrangement 40 switches to its release state. As soon as the leading tip 13 reaches the body cavity, the opposing force falls below the biasing force, thereby displacing the leading member 10 into its front position, and switching the arresting arrangement 40 back to its arresting state. Effectively, the displacement of the leading member 10 causes the manipulating member 11A to manipulate the arresting member 42 such that the arresting member 42 then interlockingly connects to the stationary member 35, thereby switching the arresting arrangement 40 into its arresting state to prevent the operation of the advancement mechanism 20. At this state, the advancement mechanism 20 cannot advance the leading tip 13 any further. Than, the device 1 can be removed, and the drainage tube, wrapped onto the drain tube mounting member 26B, can be deployed with one end thereof inside the body cavity and the other outside the body, thereby facilitating drainage of fluids from the body cavity.

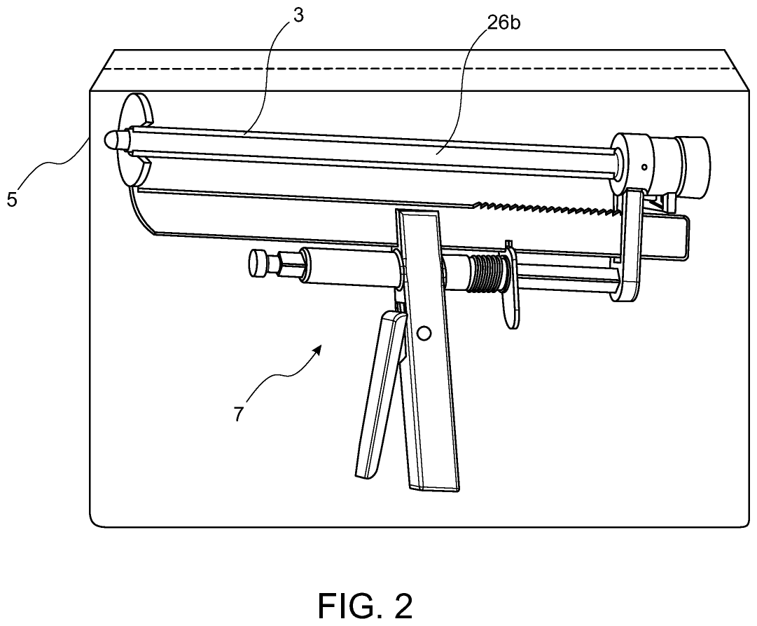

According to another aspect of the presently disclosed subject matter seen in FIG. 2, there is provided a body penetration kit comprising the handheld body penetration device 1 as described above; and a drainage tube 3 configured to be deployed between the body cavity and an exterior of the body for withdrawing fluids therefrom. The drainage tube 3 is wrapped upon the external surface of the drain tube mounting member 26B. The kit further comprises a sterile bag 5 accommodating the handheld body penetration device 1 and the drainage tube 3, optionally while the latter is mounted thereon, in a sterile manner. The kit can be a single use kit and the sterile bag 5 can be a disposable bag.

1. A handheld body penetration device for controllably penetrating through a body tissue into a body cavity, and for self arresting said penetration upon reaching said body cavity, said handheld body penetration device comprising:
a. an elongated leading member having a proximal portion, a distal portion, and a longitudinal axis extending therebetween, said distal portion having a leading tip for penetrating said body tissue;
b. an advancement mechanism operatively connected to said leading member, having an actuator configured to be actuated to induce axial advancement of the leading tip in an advancement direction along the longitudinal axis to a predetermined extent, within the body tissue; and
c. an arresting arrangement configured to prevent said axial advancement upon the leading tip reaching the body cavity.

2. The handheld body penetration device according to claim 1, wherein the advancement mechanism is configured to advance the leading tip in discrete incremental steps.

3. The handheld body penetration device according to claim 2, wherein the actuator is configured to be repeatedly actuated to advance the leading tip by a single increment with each actuation.

4. The handheld body penetration device according to claim 1, wherein the actuator is spaced apart from the leading member.

5. The handheld body penetration device according to claim 1, wherein the actuator is spaced apart from the longitudinal axis.

6. The handheld body penetration device according to claim 1, wherein the actuator is configured to displace along an actuator path upon said application of force, said actuator path being spaced from the longitudinal axis.

7. The handheld body penetration device according to claim 1, wherein the advancement mechanism comprises a handle to be held by a hand of the user, having a proximal handle portion being most proximal to the leading member, an opposite distal handle portion, and a handle axis extending therebetween, said handle axis being angled with respect to the longitudinal axis.

8. The handheld body penetration device according to claim 7, the actuator comprises a lever trigger configured to be operated by the hand of the user while holding the handle.

9. The handheld body penetration device according to claim 1, wherein the actuator is configured to be actuated by a force applied by the hand of the user, and said advancement mechanism is configured to translate said force to said axial advancement of said leading tip.

10. The handheld body penetration device according to claim 1, further comprising a stationary member configured to remain stationary with respect to the body, and a movable member including at least the leading member being movable with respect to the stationary member, during said penetration of the leading tip thereinto.

11. The handheld body penetration device according to claim 10, wherein the stationary member comprises a leaning surface configured to lean on the body during penetration of the leading tip, through which said leading member extends, at least during operation of the device.

12. The handheld body penetration device according to claim 1, wherein the arresting arrangement is switchable between a release state in which the arresting member allows the operation of the advancement mechanism and an arresting state in which the arresting arrangement arrests the operation of the advancement mechanism.

13. The handheld body penetration device according to claim 10, wherein the arresting arrangement is configured to remain at its release state as long as the leading tip is urged against a direction of its advancement by an opposing force greater than a predetermined threshold, and switch into its arresting state in response to that opposing force falling below the predetermined threshold.

14. The handheld body penetration device according to claim 12, further comprising a stationary member configured to remain stationary with respect to the body, and a movable member including at least the leading member being movable with respect to the stationary member, during said penetration of the leading tip thereinto, wherein the arresting arrangement comprises an arresting member configured to, in the arresting state of the arresting arrangement, inter-lockingly connect the movable member with the stationary member and, in the release state of the arresting arrangement, disconnect the movable member from the stationary member.

15. The handheld body penetration device according to claim 14, wherein the arresting arrangement is configured to remain at its release state as long as the leading tip is urged against a direction of its advancement by an opposing force greater than a predetermined threshold, and switch into its arresting state in response to that opposing force falling below the predetermined threshold, wherein the arresting arrangement comprises a biasing member operatively connected to the arresting member and having a biasing force constantly biasing the leading tip in the direction of the advancement of the leading tip along the longitudinal axis against said opposing force, said biasing member being switchable between a first state corresponding to the release state of the arresting arrangement, in which the opposing force is greater than the biasing force, and a second state corresponding to the arresting state of the arresting arrangement, in which the opposing force is lesser than the biasing force.

16. The handheld body penetration device according to claim 15, wherein said biasing member is connected to said leading member such that said opposing force which the leading tip encounters is transmitted through the leading member to the biasing member to cause the biasing member to switch between first and second states thereof.

17. The handheld body penetration device according to claim 1, further comprising a drain tube mounting member surrounding said leading member having an external surface configured to be wrapped with a drainage tube.

18. (canceled)

19. (canceled)

20. A body penetration kit comprising:
a. the handheld body penetration device according to claim 1; and
b. a drainage tube configured to be deployed between a body cavity and an exterior of the body for withdrawing fluids therefrom.

21. The body penetration kit according to claim 20, wherein the drainage tube is wrapped upon an external surface of the leading member.

22. The body penetration kit according to claim 20, further comprising a sterile bag accommodating the handheld body penetration device and the drainage tube in a sterile manner.
